(19) United States
(12) Patent Application Publication
(10) Pub. No.: US 2022/0409173 A1
(43) Pub. Date: Dec. 29, 2022

(54) IMPLANTABLE ULTRASONIC TRANSDUCER

(71) Applicant: BIOTRONIK SE & Co. KG, Berlin (DE)

(72) Inventors: Eckardt Bihler, Uitikon (CH); Sven Johannsen, Nuerensdorf (CH)

(73) Assignee: BIOTRONIK SE & Co. KG, Berlin (DE)

(21) Appl. No.: 17/638,454

(22) PCT Filed: Aug. 4, 2020
(86) PCT No.: PCT/EP2020/071863

§ 371 Date: Feb. 25, 2022

(30) Foreign Application Priority Data
Sep. 3, 2019 (EP) 19195188.8

(51) Int. Cl. A61B 8/00; A61B 8/12; G01H 11/08

(52) U.S. Cl. CPC A61B 8/44; A61B 8/12; G01H 11/08; B06B 1/0655

(57) ABSTRACT

The present invention relates to an implantable ultrasonic transducer comprising a first device for generating ultrasound, a second device for receiving ultrasound, and a circuit board, wherein the device for generating ultrasound is formed from a piezoelectric polymer, which is integrated in the circuit board. The invention also relates to a medical device which can be introduced into the body and comprises an ultrasonic transducer of this type.

### CROSS-REFERENCE TO RELATED APPLICATIONS

This application is the United States National Phase under 35 U.S.C. § 371 of PCT International Patent Application No. PCT/EP2020/071863, filed on Aug. 4, 2020, which claims the benefit of European Patent Application No. 19195188.8, filed on Sep. 3, 2019, the disclosures of which are hereby incorporated by reference herein in their entireties.

### TECHNICAL FIELD

The present invention relates to an implantable ultrasonic transducer and to a medical device comprising an ultrasonic transducer of this type.

### BACKGROUND

Ultrasonic transducers are already used in numerous medical applications, in particular imaging procedures. Ultrasonic transducers may also be used for other tasks, for example for measuring layer thicknesses and distances. To this end, concepts tailored to the particular task in question are required in order to provide an optimal transducer structure. Particular requirements must be met for use within the body or on the skin.

Ultrasonic transducers for diagnostics and measurement technology must generate the greatest possible sound pressure and at the same time be able to perform their detection function as sensitively and quickly as possible. This conflict requires signal processing as closely as possible to the transducer. Due to the complex structure formed of piezoceramic, semiconductor components and circuit board, the miniaturization necessary for catheters and implants is subject to limits.

Nowadays, predominantly piezoceramics formed from PZT (lead-zirconate-titanate) are used as ultrasonic transducers. These piezoceramics can be produced economically and have a strong piezoelectric effect, which results in large sound amplitudes when sound waves are generated. The disadvantages are limitations for structuring and thickness dimensions, which in turn limits the sensitivity of the detection and the signal bandwidth, as well as the possibilities for miniaturization.

A further disadvantage is the poor acoustic adjustment to bodily tissue, which leads to high energy losses by reflection at the interface between ceramic and bodily tissue. Also in view of the problematic lead content, PZT ceramics are not the first choice for catheters and implants and are only usable in catheters and implants with additional effort. The resultant costs limit the potential applications.

Further possible materials are semiconductor MEMS (CMUT—capacitive micromachined ultrasound transducer) and AlN. Among the organic materials with piezoeffect, PVDF (polyvinylidene fluoride) and its copolymer P(VDF-TrFE) stand out in particular.

Although the piezoeffect in P(VDF-TrFE) is only 1/10 of the value of PZT, the acoustic impedance of PVDF, at 4.2 MPa*s/m, is only slightly above that of bodily tissue (1.6 MPa*s/m) and is much better adapted than PZT, at 30 MPa*s/m, to water or bodily tissue. Since PVDF is available in films with thicknesses from 10 μm, very sensitive detectors with high bandwidth can be produced. PVDF is therefore used in particular for hydrophones (ultrasonic detectors for underwater applications).

The present disclosure is directed toward overcoming one or more of the above-mentioned problems, though not necessarily limited to embodiments that do.

### SUMMARY

Based on this background, it is in particular an objective of the present invention to provide a reliable and precise ultrasonic transducer which has a simplified structure and can be used in a patient's body, and also corresponding medical devices that can be introduced into the body.

At least this objective is achieved by an implantable ultrasonic transducer having the features of claim 1 and a medical device having the features of claim 14. Advantageous embodiments are described in the dependent claims and the following description.

According to claim 1, an implantable ultrasonic transducer is provided. The ultrasonic transducer comprises:
a first device for generating ultrasound, also referred to here as an ultrasound generator,
a second device for receiving ultrasound, also referred to here as an ultrasound receiver, and
a circuit board.

In accordance with the present invention, it is particularly envisioned that the second device for receiving ultrasound is formed from a piezoelectric polymer, which is integrated in the circuit board.

The term “piezoelectric polymer” in the context of the present invention relates in particular to a polymer which is characterized by a piezoelectric effect, i.e. the occurrence of a voltage in the event of elastic deformation.

The term “circuit board” in the context of the present invention is understood in the sense of its meaning known to the person skilled in the art. A “circuit board” refers in particular to a carrier of electronic components which comprises, besides an electrically insulating substrate, also conductor tracks for electrically contacting electronic components.

By separating the ultrasound generator and ultrasound receiver in two separate devices, the design of the ultrasound receiver can advantageously be simplified and made biocompatible in a simple manner. This is achieved in particular by integrating the ultrasound receiver in the circuit board, which itself is preferably fabricated from a biocompatible material.

Accordingly, in accordance with one embodiment of the ultrasonic transducer according to the present invention, the circuit board comprises or consists of a liquid-crystal polymer.

In accordance with one embodiment of the implantable ultrasonic transducer according to the present invention, the second device for receiving ultrasound is formed from a plurality of piezoelectric elements formed from the piezoelectric polymer, wherein the plurality of piezoelectric elements are integrated in the circuit board.

The piezoelectric elements here may assume any desired form, for example substantially cylindrical, cube-shaped, cuboidal or prism-shaped.

In accordance with a further embodiment of the implantable ultrasonic transducer according to the present invention, the piezoelectric elements are arranged in the circuit board in an array, for example 3×3, 5×5, 10×10 or 20×50. Such an array advantageously allows a spatially resolved measurement of the sound pressure, in particular of the sound reflected by bodily tissues.

In accordance with a further embodiment of the implantable ultrasonic transducer according to the present invention, the circuit board has a thickness in the range of from 0.01 mm to 0.1 mm.

In accordance with a further embodiment of the implantable ultrasonic transducer according to the present invention, the piezoelectric elements, independently of one another, have a length in the range of 0.01 mm to 5 mm.

In accordance with a further embodiment of the implantable ultrasonic transducer according to the present invention, the first device for generating ultrasound is formed from a piezoelectric material, in particular a ceramic or a crystal, preferably a PZT (lead-zirconate-titanate) ceramic.

In accordance with a further embodiment of the implantable ultrasonic transducer according to the present invention, the piezoelectric material has a thickness in the range of 0.1 mm to 2 mm.

In accordance with a further embodiment of the implantable ultrasonic transducer according to the present invention, the first device for generating ultrasound is arranged at or on the circuit board.

The first device for generating ultrasound is advantageously connected to the circuit board via an adhesive layer. The adhesive layer may be electrically conductive all over or only at certain points. Non-limiting examples of suitable adhesives include epoxy or acrylic resins which are filled with electrically conductive particles formed, for example, from metal or carbon. Such adhesives comprise, in particular, up to 30 vol. % of electrically conductive particles.

In accordance with a further embodiment of the implantable ultrasonic transducer according to the present invention, the first device for generating ultrasound and the second device for receiving ultrasound are electrically contactable independently of one another. The structure, in particular the circuit, of the ultrasonic transducer according to the present invention may thus be simplified in that the generation and the detection are performed in circuits that are separate from one another. An improved signal-to-noise ratio or a greater sensitivity of the detection function may be achieved with the separation of sound generation and detection.

In accordance with a further embodiment of the implantable ultrasonic transducer according to the present invention, the circuit board comprises at least one electrical conductor track, which electrically contacts the second device for receiving ultrasound.

In accordance with a further embodiment of the implantable ultrasonic transducer according to the present invention, the first device, in particular the piezoelectric material for generating ultrasound comprises a metal layer on the upper side and the bottom side. The metal layer on the upper side may be used advantageously as ground for the ultrasound receiver.

In accordance with a further embodiment of the implantable ultrasonic transducer according to the present invention, the first device for generating ultrasound is arranged with the upper side at the circuit board, wherein the metal layer on the upper side electrically contacts the device for receiving ultrasound, in particular directly or indirectly via an electrically conductive adhesive layer.

In accordance with an alternative embodiment of the implantable ultrasonic transducer according to the present invention, the first device for generating ultrasound comprises a metal layer on each of two mutually opposed side faces or end faces. With an ultrasonic transducer designed in this way, it is advantageously possible to couple transverse waves into the surrounding environment, for example bodily tissue.

In accordance with a further embodiment of the implantable ultrasonic transducer according to the present invention, the circuit board is flexible. It is thus advantageously possible to adapt the circuit board to the form or contour of the ultrasonic generator.

In accordance with a further embodiment of the implantable ultrasonic transducer according to the present invention, the piezoelectric material is designed substantially in the form of a hollow cylinder, wherein the outer side (upper side) of the hollow cylinder comprises a metal layer, the surface (bottom side) delimiting the aperture through the hollow cylinder is coated with metal or the aperture through the hollow cylinder is filled with metal, and wherein the circuit board substantially fully surrounds the metal layer on the outer side. It is advantageously possible to generate a cylindrical wave with this embodiment of the implantable ultrasonic transducer according to present invention. In particular, vessels such as blood vessels can thus be examined by ultrasound. This embodiment is thus suitable in particular for use in a catheter.

In accordance with a further embodiment of the implantable ultrasonic transducer according to the present invention, the piezoelectric polymer is polyvinylidene fluoride or a copolymer thereof.

In accordance with a further embodiment of the implantable ultrasonic transducer according to the present invention, the implantable ultrasonic transducer is coated at least in part with a biocompatible coating or polymer, in particular polydimethylsiloxane (PDMS) or a polyurethane. Any exposed conductor tracks of the circuit board may thus preferably be electrically insulated with respect to bodily tissue or bodily fluids and/or protected against corrosion.

According to claim 14, a medical device that can be introduced, in particular implanted, into the body is provided, which comprises the implantable ultrasonic transducer according to the present invention.

In a further embodiment, the medical device according to the present invention that can be introduced, in particular implanted, into the body is designed as an active implant, as a sensor, as a loop recorder or as a catheter.

The term “loop recorder” in the sense of the present invention refers in particular to a passive implant which measures or monitors physiological parameters of a patient, for example the electrical activity of the heart.

In a further embodiment, the medical device according to the present invention that can be introduced, in particular implanted, into the body is designed as a pacemaker, cardioverter-defibrillator, neurostimulator or muscle stimulator.

In a further embodiment, the ultrasonic transducer according to the present invention is arranged in the medical device formed as a catheter, moreover at the distal end of the catheter. The piezoelectric material is preferably formed here substantially in the form of a hollow cylinder, wherein the outer side (upper side) of the hollow cylinder comprises a metal layer, the surface (bottom side) delimiting the aperture through the hollow cylinder is coated with metal or the aperture through the hollow cylinder is filled with metal, and wherein the circuit board substantially fully surrounds the metal layer on the outer side. The medical device formed as a catheter preferably also comprises a catheter tube, one or more guide wires, and one or more electronic components, in particular for processing signals from the ultrasound receiver, which in particular are mounted on the circuit board.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further features and advantages of the present invention will be explained hereinafter with reference to the figure description of exemplary embodiments. The figures show:

FIGS. 1-6 show various embodiments of the implantable ultrasonic transducer according to the present invention.

### DETAILED DESCRIPTION

A significant concept of the present invention is the separation of the detection of the sound generation in ultrasonic transducers. Only a thin layer is used for the detection and is fixedly connected on a sound generator.

A further inventive concept is the integration of the piezoelectric elements that are used for the detection in a flexible circuit board. The detectors are thus directly electrically attached and may be directly connected to electric components without additional mounting and connection technology.

The present invention allows a further miniaturization and simplification in the structure with much lower costs, and therefore further applications are possible. A further advantage is the use of biocompatible materials.

In particular, as a result of the present invention, the construction of ultrasonic transducers for catheters and implants is considerably simplified and may be further miniaturized. The costs of the connection technology can be considerably reduced.

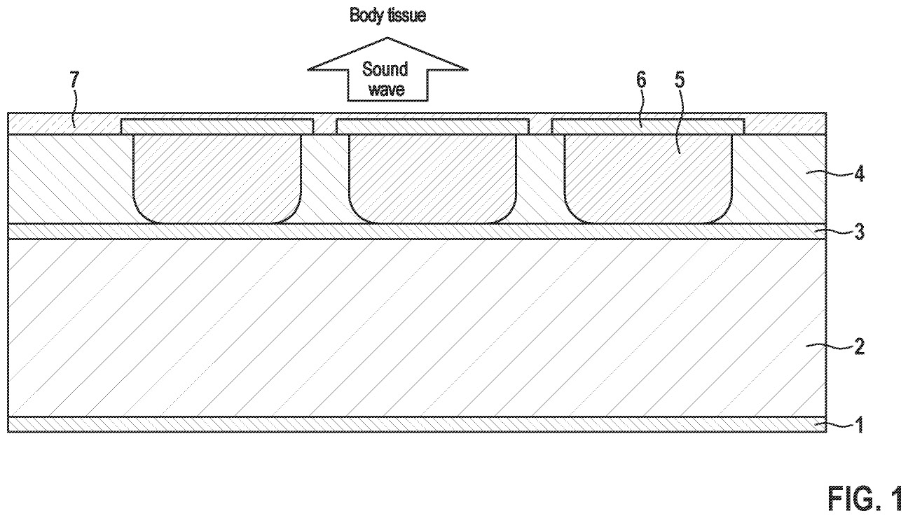

FIG. 1 shows an embodiment of the implantable ultrasonic transducer according to the present invention. A thin polymer film 4 is applied to a sound generator formed from a piezoelectric material 2, which is coated on the upper side 3 and the bottom side 1 with a metal layer. In the simplest case, this polymer film is formed from PVDF (polyvinylidene difluoride) or P(VDF-TrFE) (poly(vinylidene fluoride-co-trifluoroethylene), which is made piezoelectric by suitable polarisation. This polymer film, however, is preferably a flexible circuit board which for example is formed from polyimide or LCP (liquid crystal polymer) or another base material for flexible circuit boards. This has the advantage that the flexible circuit board already has electrical conductor tracks which are connected in a suitable way to the metal layers 1, 3 and 6. Due to these conductor tracks, the piezoelectric materials 2 and 5 are electrically connected to a suitable electrical circuit for generating pulses for sound generation and for signal processing in the detection of reflected sound signals from the bodily tissue. In this case, for example, the polymer P(VDF-TrFE) 5 is introduced into suitable indentations 5 in the flexible circuit board 4. This can be achieved either by way of a heating process (P(VDF-TrFE) is thermoplastic and melts at approximately 150° C.) or by applying a highly viscous solution of P(VDF-TrFE) in a suitable solvent by way of screen printing or using a doctor blade. Before applying the metallization 6, the P(VDF-TrFE) is polarised by means of corona discharge in order to make it piezoelectric. The metal structure for deriving the signals is then applied and structured. This can be achieved by means of sputtering and lift-off. Lastly, the entire transducer is coated with a thin (biocompatible) coating 7 or with silicone (PDMS) 7 so that the conductor tracks 6 are not short-circuited by the bodily tissue.

In this case, the components of the ultrasonic transducer according to the present invention preferably have the following dimensions:
thickness of the sound generator 2: 0.1 mm to 2 mm
thickness of the polymer film/flexible circuit board 4: 0.01 mm to 0.1 mm
dimensions of the piezoelectric elements 5: 0.01 mm to 5 mm

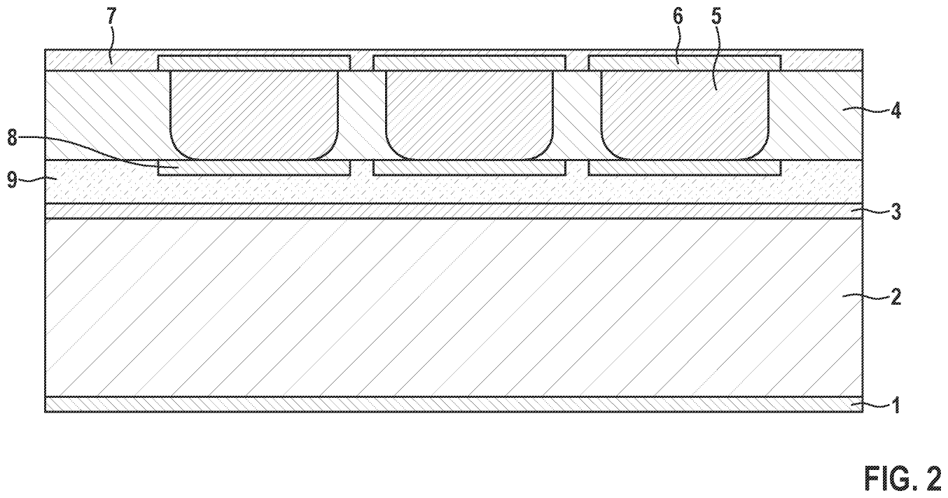

In the embodiment of the ultrasonic transducer according to the present invention shown in FIG. 2, the flexible circuit board consisting of the base material 4, the piezoelectric elements 5, and the conductor tracks 6 and 8 are first produced. This can be implemented in a production format typical for circuit boards (for example 300×450 mm or also even larger). The sound generator 2 is then glued to the rear face of the circuit board. The adhesive layer 9 may be electrically conductive all over or only at specific points, and therefore electrical contact for connecting the sound generator 2 to one of the conductor tracks 8 of the flexible circuit board is created.

This structure has advantages in particular if the circuit board also has other functions and the sound generator and the detector form only part of the circuit board.

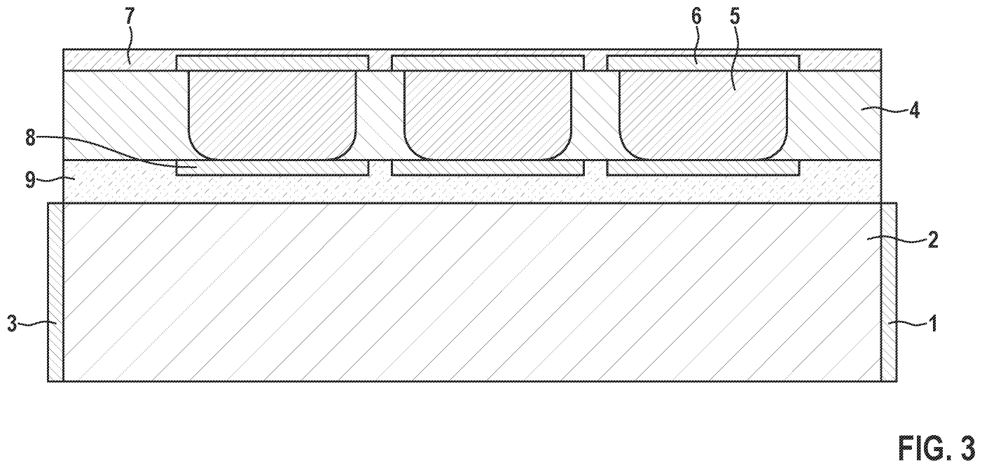

If the sound generator 2 has electrical contacts at the end faces, as shown in FIG. 3, a higher component of transverse waves can be coupled into the bodily tissue. Such an arrangement is only possible if the detector and the sound generator are separate.

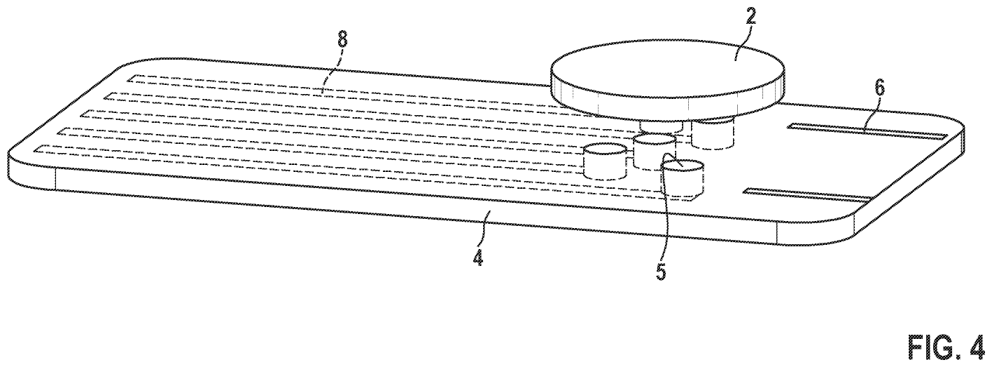

The full structure is shown schematically in FIG. 4. The piezoelectric elements 5 are integrated in the circuit board 4 and connected via the conductor tracks 8. The conductor tracks 6 running on the upper side are only indicated schematically. The sound generator 2 is adhesively bonded onto the circuit board. The order of the illustration is reversed here as compared to the cross sections.

This arrangement may be integrated directly in a device. The circuit board can also be applied directly to the skin or can be mounted on the tip or the balloon of a catheter. Sound-absorbent materials can be mounted on the upper side of the sound generator 2 in order to achieve a higher energy transfer in the other direction towards the bodily tissue. Suitable sound-absorbent materials are polymers in general and elastomers in particular.

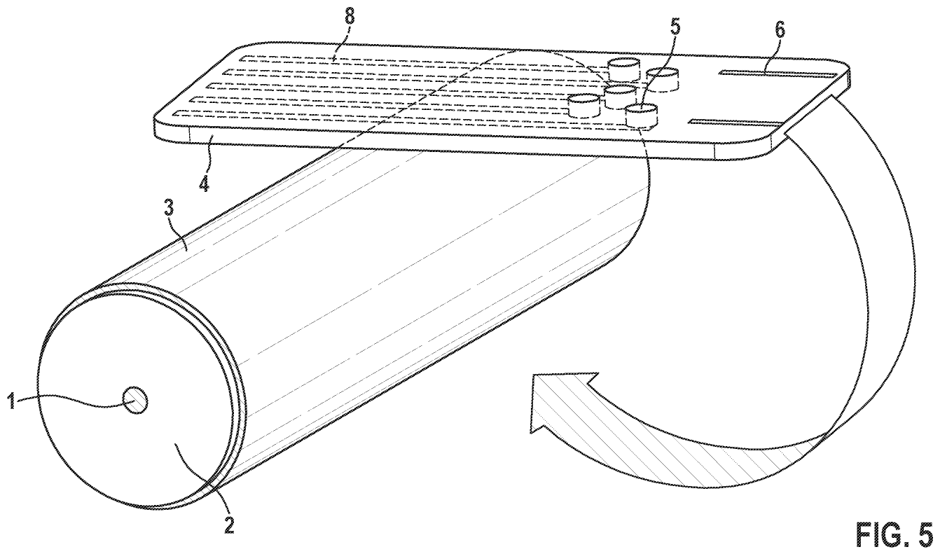

In another embodiment of the ultrasonic transducer according to the present invention shown in FIG. 5, the circuit board 4 with the integrated detectors or piezoelectric elements 5 is glued over a cylindrical sound generator 2.

A cylindrical transducer is used in the case of this concept. A metal face 1 is in this case formed as a wire in the centre of the piezoelectric material 2. The outer face of the cylinder 2 is metallized 3. In the simplest case, the metallization 3 is provided on the bottom side of the detector film or circuit board 4.

With the embodiment shown in FIG. 5, a cylindrical wave may advantageously be produced, which allows improved reflection behaviour in the vessel and thus a more accurate measurement. This arrangement is therefore of interest in particular for use in catheters.

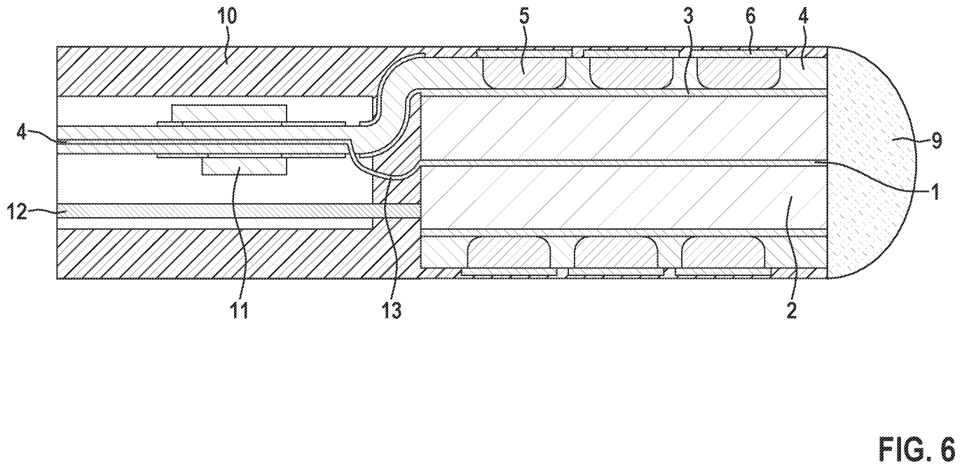

A catheter designed in such a way is shown schematically in FIG. 6. The catheter in this case comprises, at its distal end, the ultrasonic transducer according to the present invention comprising a flexible circuit or circuit board 4 with integrated piezoelectric elements 5 as ultrasound receiver and a coaxial cylindrical ultrasound generator 2. The catheter furthermore comprises a tip 9 (as mechanical termination without further function), a catheter tube 10, electronic components 11, one or more guide wires 12 (guide wire), and an insulated wire 13 for electrically contacting the ultrasound generator 2 (preferably with high voltage). The wire 13 can be connected here to the flexible circuit board 1 or can be guided separately by the catheter tube 10. The electronic components 11 are designed here in particular for the signal processing of the ultrasound receiver 4, 5 and can preferably be mounted on the flexible circuit board 4. Such a catheter preferably has a diameter in the range of from 1.5 mm to 4 mm.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable ultrasonic transducer comprising
a first device for generating ultrasound,
a second device for receiving ultrasound, and
a circuit board,
wherein
the second device for receiving ultrasound is formed from a piezoelectric polymer, which is integrated in the circuit board.

2. The implantable ultrasonic transducer according to claim 1, wherein the second device for receiving ultrasound is formed from a plurality of piezoelectric elements formed from the piezoelectric polymer, wherein the plurality of piezoelectric elements are integrated in the circuit board.

3. The implantable ultrasonic transducer according to claim 1, wherein the first device for generating ultrasound is formed from a piezoelectric material, in particular a ceramic or a crystal.

4. The implantable ultrasonic transducer according to claim 1, wherein the first device for generating ultrasound is arranged at the circuit board.

5. The implantable ultrasonic transducer according to claim 1, wherein the first device for generating ultrasound and the second device for receiving ultrasound are electrically contactable independently of one another.

6. The implantable ultrasonic transducer according to claim 1, wherein the circuit board comprises at least one electrical conductor track, which electrically contacts the second device for receiving ultrasound.

7. The implantable ultrasonic transducer according to claim 1, wherein the first device for generating ultrasound has a metal layer on each of the upper side and the bottom side.

8. The implantable ultrasonic transducer according to claim 7, wherein the first device for generating ultrasound is arranged with the upper side on the circuit board, wherein the metal layer on the upper side electrically contacts the second device for receiving ultrasound.

9. The implantable ultrasonic transducer according to claim 1, wherein the first device for generating ultrasound has a metal layer on each of two mutually opposed side faces.

10. The implantable ultrasonic transducer according to claim 1, wherein the circuit board is flexible.

11. The implantable ultrasonic transducer according to claim 3, wherein the piezoelectric material is designed substantially in the form of a hollow cylinder, wherein the outer side of the hollow cylinder comprises a metal layer, the surface delimiting the aperture through the hollow cylinder is coated with metal or the aperture through the hollow cylinder is filled with metal, and wherein the circuit board substantially fully surrounds the metal layer on the outer side.

12. The implantable ultrasonic transducer according to claim 1, wherein the piezoelectric polymer comprises or consists of polyvinylidene fluoride or a copolymer thereof.

13. The implantable ultrasonic transducer according to claim 1, wherein the implantable ultrasonic transducer is coated at least in part with a biocompatible coating or polymer comprising a polydimethylsiloxane or a polyurethane.

14. A medical device that can be introduced, in particular implanted, in the body, comprising an implantable ultrasonic transducer according to claim 1.

15. The medical device according to claim 14 that can be introduced in the body, wherein the medical device is formed as an active implant, a sensor, a loop recorder or a catheter.
